(19) United States
(12) Patent Application Publication
(10) Pub. No.: US 2024/0424133 A1
(43) Pub. Date: Dec. 26, 2024

(54) mRNA THERAPY WITH REDUCED IMMUNE REACTOGENICITY

(71) Applicants: RVAC Medicines (US), Inc., Waltham, MA (US); Acuitas Therapeutics Inc., Vancouver (CA)

(72) Inventors: Isabel M. CHU, Waltham, MA (US); Yang XIANG, Waltham, MA (US); Stefano GULLA, Waltham, MA (US); Jason J. ZHANG, Waltham, MA (US); Paulo Jia Ching LIN, Vancouver (CA); Ying K. TAM, Vancouver (CA)

(21) Appl. No.: 18/421,751

(22) Filed: Jan. 24, 2024

Related U.S. Application Data
(60) Provisional application No. 63/481,225, filed on Jan. 24, 2023.

(51) Int. Cl. A61K 48/00; A61K 9/51; A61K 45/06; C07K 16/00

(52) U.S. Cl. CPC A61K 48/0008; A61K 9/5123; A61K 45/06; C07K 16/00; C07K 2317/94

(57) ABSTRACT

The present disclosure provides compositions and methods for mRNA therapy with reduced immune reactogenicity.

### FIELD OF DISCLOSURE

The present disclosure provides compositions and methods for mRNA therapy including mRNA constructs encoding therapeutic agents with reduced immune reactogenicity.

### BACKGROUND

Messenger RNA therapy (referred to herein as “mRNA”) is a cutting-edge therapeutic modality, and a current approach is to encapsulate an mRNA construct in lipid nanoparticles (LNPs) and deliver the LNP-mRNA into cells to produce a protein encoded by the mRNA construct. This therapy has the potential to treat a wide range of diseases, for example but not limited to, cancer, autoimmune diseases, infectious diseases, and rare diseases. One of the key limitations of the applications of mRNA therapy is the immune reactogenicity of the mRNA constructs as well as the delivery vehicles such LNPs. Immune reactogenicity refers to the ability of a substance that causes an unwanted or adverse immune reaction in a subject.

In order to increase tolerable doses and/or potential for repeated dosing of mRNA therapy, compositions and methods for reducing immune reactogenicity of mRNA therapy are desirable.

### SUMMARY

The present disclosure provides compositions and methods for mRNA therapy, e.g., LNP-based mRNA therapy, with reduced immune reactogenicity.

In an aspect, the present disclosure provides a composition comprising an immunomodulating agent and a product comprising an mRNA construct encoding a therapeutic agent (referred to herein as “mRNA product”), optionally with the mRNA construct encapsulated in lipid nanoparticles.

In some embodiments of the composition disclosed herein, the immunomodulating agent reduces neutrophil expansion.

In some embodiments of the composition disclosed herein, the immunomodulating agent reduces lymphocyte trafficking.

In some embodiments of the composition disclosed herein, the immunomodulating agent reduces monocyte trafficking.

In some embodiments of the composition disclosed herein, the immunomodulating agent blocks the function of interleukin-6 (IL-6) function and/or reduces the expression interleukin-6 (IL-6) in a cell.

In some embodiments of the composition disclosed herein, the therapeutic agent is an antibody. In some embodiments, the antibody comprises a constant region which is engineered to improve the half-life of the antibody, e.g., in vivo half-life of the antibody.

In an aspect, the present disclosure provides a method comprising administering to a subject any of the composition described herein.

In an aspect, the present disclosure provides a method comprising administering to a subject a product comprising an mRNA construct encoding a therapeutic agent, e.g., a lipid nanoparticle encapsulating the mRNA construct, and administering to the subject an immunomodulating agent.

In some embodiments of the method disclosed herein, the immunomodulating agent reduces neutrophil expansion.

In some embodiments of the method disclosed herein, the immunomodulating agent reduces lymphocyte trafficking.

In some embodiments of the method disclosed herein, the immunomodulating agent reduces monocyte trafficking.

In some embodiments of the method disclosed herein, the immunomodulating agent blocks the function of interleukin-6 (IL-6) function and/or reduces the expression interleukin-6 (IL-6) in a cell.

In some embodiments of the method disclosed herein, the therapeutic agent is an antibody. In some embodiments, the antibody comprises a constant region which is engineered to improve the half-life of the antibody, e.g., in vivo half-life of the antibody.

In some embodiments of the method disclosed herein, the mRNA product, e.g., the lipid nanoparticle encapsulating the mRNA construct, and the immunomodulating agent are administered simultaneously or sequentially.

In some embodiments of the method disclosed herein, the method further comprises measuring blood cell count, neutrophil count, circulating lymphocytes count, circulating monocytes count, and/or IL-6 level of the subject. In some embodiments, the measuring is conducted before and after administration of the mRNA product (e.g., the lipid nanoparticle encapsulating the mRNA construct) and/or the immunomodulating agent.

In an aspect, the present disclosure provides a kit comprising (a) a composition which comprises an immunomodulating agent and (b) an mRNA product which comprises an mRNA construct encoding a therapeutic agent (e.g., a lipid nanoparticle encapsulating the mRNA construct).

### BRIEF DESCRIPTION OF FIGURES

FIG. 1 shows an overall study design as further described in Example 1.

FIGS. 2A-2E shows screening results of cynomolgus monkeys using an ex vivo cytokine release assay as further described in Example 2 (cytokine measurement of treated cynomolgus PBMC: IFN-gamma (FIG. 2A), IP-10 (FIG. 2B), TNF-a (FIG. 2C), IL-1b (FIG. 2D) and IL-6 (FIG. 2E)).

FIGS. 3A and 3B shows the results of while blood cell counting as further described in Example 3.

FIGS. 4A-4F shows changes in white blood cells subsets in mRNA/LNP dosed animals as further described in Example 4.

FIGS. 5A and 5B shows the serum IL-6 levels in the cytokine analysis as further described in Example 5.

FIGS. 6A and 6B shows serum hIgG titer levels of animals dosed with 10 mg/kg WT or YTE recombinant antibody or with 2.5 mg/kg mRNA coated LNP expressing the WT or YTE antibody.

### DETAILED DESCRIPTION

### Definitions

The disclosures and embodiments set forth herein are to be construed as exemplary only and not as limiting the scope of the invention. Although specific terms are employed herein, unless otherwise noted, they are used in a generic and descriptive sense only and not for purposes of limitation.

All publications, patents, and patent applications referred to herein are incorporated by reference in their entirety to the same extent as if each individual publication, patent, or patent application was specifically and individually indicated to be incorporated by reference in its entirety.

In the present disclosure, unless otherwise specified, the scientific and technical terms used herein have the meanings generally understood by a person skilled in the art. Although any methods and materials similar or equivalent to those described herein find use in the practice of the present disclosure, the preferred methods and materials are described herein. Accordingly, the terms defined herein are more fully described by reference to the Specification as a whole.

Unless defined otherwise, all technical and scientific terms, acronyms, and abbreviations used herein have the same meaning as commonly understood by one of ordinary skill in the art to which the disclosure pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present disclosure also contemplates that in some embodiments of the disclosure, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

It is to be understood that this disclosure is not limited to the particular methodology, protocols, and reagents described, as these may vary, depending upon the context in which they are used by those skilled in the art.

As used herein, a composition refers to any mixture of two or more products, substances, or compounds, including cells.

As used herein, the term “RNA” or “ribonucleic acid” refers to a biomolecule composed of a chain of ribonucleotides, which are molecules made of a nitrogenous base, a sugar, and a phosphate group. RNA molecules include, but are not limited to, messenger RNA (mRNA), transfer RNA (tRNA), ribosomal RNA (rRNA), and small non-coding RNA (ncRNA) such as microRNA (miRNA) and small interfering RNA (siRNA). As used herein, “mRNA” or “messenger RNA” refers to an RNA molecule that comprises a protein-encoding sequence, which can be translated by ribosome in the process of protein synthesis.

As used herein, the term “lipid nanoparticle” refers to a liposomal transfer vehicle with at least one dimension in the nanometer scale which comprises one or more lipids (e.g., cationic lipids, non cationic lipids, cholesterol, and PEG-modified lipids).

Delivery of mRNA Therapy

mRNA can be delivered into cells by various means, such as liposome complexes, polymers (e.g., poly-amido-amine (PAA), poly-beta amino-esters (PBAEs) and polyethylenimine (PEI)), ferritin nanoparticles, platelet membrane-coated nanoparticles, and liposome nanoparticles. Liang, Y. et al., Development and Delivery Systems of mRNA Vaccines, Front. Bioeng. Biotechnol., 27 Jul. 2021, Volume 9—2021, https://doi.org/10.3389/fbioe.2021.718753.

Lipid nanoparticle (LNP)-based mRNA therapy (referred to as “LNP-mRNA”) is a cutting-edge therapeutic modality in which messenger RNA (mRNA) is encapsulated in the LNP and delivered into cells to produce the encoded protein. This therapy has the potential to treat a wide range of diseases, for example but not limited to, cancer, autoimmune diseases, infectious diseases, and rare diseases.

mRNA therapy is a powerful tool, which can theoretically produce any protein/peptide via the protein synthesis machine processed in the transfected cell in vitro or in vivo. To achieve therapeutic effects, mRNA molecules have to reach specific target cells and produce sufficient proteins of interest. Lipid nanoparticle (LNP) is a safe and effective delivery vehicle of mRNA.

Lipid nanoparticle is a liposomal transfer vehicle comprising one or more lipids (e.g., cationic lipids, non cationic lipids, cholesterol, and PEG-modified lipids). LNP-mRNA formulations manufactured by rapid mixing exhibit a stable nanostructure, in which mRNA molecules can be encapsulated in the interior core through electrostatic interactions with the lipids. This structural feature protects mRNA molecules from nuclease degradation and increases nanoparticle stability in physiological fluids. In some embodiments, incorporating PEG-lipids further decreases recognition by the mononuclear phagocyte system and clearance by renal filtration. Additionally, targeted biodistribution of LNP-mRNA formulations can be improved by further modifying and optimizing the nanoparticle, for example, by coating the LNPs with antibodies to deliver mRNA molecules into inflammatory leukocytes and epidermal growth factor receptor (EGFR)-positive tumour cells for treating inflammatory bowel disease and cancer, respectively. Organ selectivity can also be achieved by adjusting the proportions of lipid components, for example, to design spleen-targeted mRNA vaccines or lung-targeted genome editing delivery systems. (Kedmi, R. et al. A modular platform for targeted RNAi therapeutics. Nat. Nanotechnol. 13, 214-219 (2018); Veiga, N. et al. Cell specific delivery of modified mRNA expressing therapeutic proteins to leukocytes. Nat. Commun. 9, 4493 (2018); Kranz, Lena M., et al. “Systemic RNA delivery to dendritic cells exploits antiviral defence for cancer immunotherapy.” Nature 534.7607 (2016): 396-401; Liu, S. et al. Membrane-destabilizing ionizable phospholipids for organ-selective mRNA delivery and CRISPR-Cas gene editing. Nat. Mater. 20, 701-710 (2021)) Once they reach target cells, lipid nanoparticles can be internalized by multiple mechanisms, including macropinocytosis and clathrin-mediated and caveolae-mediated endocytosis. Positively charged lipids may facilitate electrostatic interaction and fusion with negatively charged endosomal membranes, resulting in the leak of mRNA molecules into the cytoplasm.

One of the key limitations of LNP-mRNA's application is the immune reactogenicity of LNP-mRNA. Immune reactogenicity refers to the ability of a substance to cause an unwanted or adverse immune reaction in the body. As used herein, the immune reactogenicity of an LNP-mRNA formulation refers to the unwanted or adverse immune reaction caused by the mRNA-encapsulated LNP molecules. The formulation of LNP and the composition of the encapsulated mRNA may impact the immune reactogenicity of an LNP-mRNA formulation.

In order to increase tolerable doses and/or potential for repeated dosing of an LNP-mRNA therapy, compositions and methods for reducing immune reactogenicity of an LNP-mRNA therapy are desirable.

The present disclosure discovered multiple in vivo biomarkers indicating immune activation after LNP-mRNA intervention. Examples 1-5. In these examples, differences were measured in pharmacodynamics and pharmacokinetic responses between recombinant human IgG1 and LNP-mRNA encoded human IgG1. Five in vivo biomarkers for immune reactogenicity event caused by LNP-mRNA molecules were identified. These in vivo biomarkers include (1) increased blood cell count, (2) increased neutrophil count, (3) decreased circulating lymphocyte count, (4) decreased circulating monocyte count, and (5) increased interleukin-6 (IL-6) level in circulation. Given these in vivo biomarkers of immune activation by LNP-mRNA, the present disclosure provides compositions and methods for LNP-mRNA therapy with reduced immune reactogenicity, by combining one or more immunomodulating agent with the LNP-mRNA. Specifically, in some embodiments, the immunomodulating agent reduces neutrophil expansion, reduces lymphocyte trafficking, reduces monocyte trafficking, blocks the function of IL-6, and/or reduces IL-6 expression in a subject.

### Compositions and Kits

In an aspect, the present disclosure provides a composition comprising an immunomodulating agent and a product comprising an mRNA construct encoding a therapeutic agent, e.g., a lipid nanoparticle encapsulating the mRNA construct. In some embodiments, the immunomodulating agent is in a form of a protein. In some embodiments, the immunomodulating agent is in a form of a nucleic acid. In some embodiments, the composition comprises a lipid nanoparticle which comprises an mRNA encoding a therapeutic agent and an mRNA encoding the immunomodulating agent. In some embodiments, the composition comprises a lipid nanoparticle which comprises an mRNA encoding a therapeutic agent and encoding the immunomodulating agent.

In another aspect, the present disclosure provides a kit comprising (a) a composition which comprises an immunomodulating agent and (b) a product comprising an mRNA construct encoding a therapeutic agent, e.g., a lipid nanoparticle encapsulating the mRNA construct.

As used herein, the term “immunomodulating agent” refers to a substance that stimulates or suppresses the immune system of a subject. Specific immunomodulating agents, such as monoclonal antibodies, cytokines, and vaccines, affect specific parts of the immune system. Nonspecific immunomodulating agents, such as BCG, affect the immune system in a general way. Some examples of immunomodulating agents include immunosuppressants, which are used to suppress the immune response in conditions such as autoimmunity and transplant rejection, and immunostimulants, which are used to enhance immune function in conditions such as cancer and chronic infections. Some exemplary immunomodulating agents are cytokines, interferons, interleukins, and BCG.

In some embodiments of the composition disclosed herein, the immunomodulating agent suppresses the immune system. In some embodiments, the immunomodulating agent is a specific suppressive immunomodulating agent.

In some embodiments of the composition disclosed herein, the immunomodulating agent reduces neutrophil expansion. Neutrophils are a type of white blood cell (leukocytes). There are three types of white blood cells: granulocytes, lymphocytes, and monocytes. Neutrophils are a subset of granulocytes, along with eosinophils and basophils cells. Neutrophils are the most abundant leukocytes in the circulation and have been regarded as first line of defense in the innate arm of the immune system. They capture and destroy invading microorganisms, through phagocytosis and intracellular degradation, release of granules, and formation of neutrophil extracellular traps after detecting pathogens. Neutrophils also participate as mediators of inflammation. As such, changes in their relative and absolute amounts serve as sensitive markers of immune activation. As used herein, “neutrophil expansion” can be manifested in an increase in the count of neutrophil (e.g., measured by count in thousands of cells per microliter of blood, k/ul). In some embodiments, the immunomodulating agent can impede or inhibit neutrophil expansion.

In some embodiments of the composition disclosed herein, the immunomodulating agent reduces lymphocyte trafficking. Lymphocyte is a type of immune cell that is made in the bone marrow and is found in the blood and in lymph tissue. Lymphocytes include natural killer cells (which function in cell-mediated, cytotoxic innate immunity), T cells (for cell-mediated, cytotoxic adaptive immunity), and B cells (for humoral, antibody-driven adaptive immunity). Lymphocytes are migratory cells, trafficking from their sites of origin in the bone marrow and thymus and homing to and recirculating through specialized lymphoid and extra lymphoid tissues in the periphery. In order to protect the body, lymphocytes must be able to access the many sites where pathogens may appear. Toward this end they continually travel through the body by way of the circulatory and lymphatic systems. The circulatory system provides rapid access to bodily tissues, but is more than a mere highway along which lymphocytes travel. The lymphatic system, which ultimately channels lymph back into the circulation via the thoracic duct, provides a route for lymphocytes that have exited the bloodstream to return and circulate anew. Antigen-inexperienced lymphocytes patrolling for foreign substances can thus continuously migrate from blood to secondary lymphoid organ (SLO) to lymph and back to blood again. By the same token, antigen-experienced effector lymphocytes can reach infected tissues to help neutralize and eliminate the pathogen(s), while memory cells can return to previously infected sites to guard against pathogen reappearance. Primary lymphoid organs include thymus, and bone marrow. SLO include peripheral lymph nodes, peye's patches, mesenteric lymph nodes, and spleen.

For the purpose of this application, the degree of lymphocyte trafficking can be measured by the count of lymphocyte in a subject's blood (e.g., measured by count in thousands of cells per microliter of blood, k/ul). Thus, reducing lymphocyte trafficking can be achieved by reducing lymphocyte count in blood.

In some embodiments of the composition disclosed herein, the immunomodulating agent reduces monocyte trafficking. Monocyte is a type of immune cell that is made in the bone marrow and travels through the blood to tissues in the body where it becomes a macrophage or a dendritic cell. Macrophages surround and kill microorganisms, ingest foreign material, remove dead cells, and boost immune responses. During inflammation, dendritic cells boost immune responses by showing antigens on their surface to other cells of the immune system. Monocyte recruitment is guided by chemokines that bind to receptors expressed on the monocyte cell surface. The process follows a general paradigm of leukocyte adhesion and trafficking, and thus depends on the interactions of various adhesion molecules. Circulating monocytes traffic into tissues during both homeostasis and inflammation. When conditioned by local growth factors, pro-inflammatory cytokines and microbial products, monocytes can differentiate into macrophage or dendritic cell populations. The recruitment of monocytes is essential for effective control and clearance of bacterial, protozoal, fungal and viral infections, but recruited monocytes can also be deleterious and cause immunopathology during certain infections.

For the purpose of this application, the degree of monocyte trafficking can be measured by the count of monocyte in a subject's blood (e.g., measured by count in thousands of cells per microliter of blood, k/ul). Thus, reducing monocyte trafficking can be achieved by reducing lymphocyte count in blood.

In some embodiments of the composition disclosed herein, the immunomodulating agent blocks the function of interleukin-6 (IL-6) function and/or reduces the expression interleukin-6 (IL-6) in a cell. IL-6 is a pleiotropic, proinflammatory cytokine produced by a variety of cell types, including lymphocytes, monocytes, and fibroblasts. IL-6 functions as a mediator for notification of the occurrence of some emergent event. IL-6 is generated in an infectious lesion and sends out a warning signal to the entire body. IL-6 also issues a warning signal in the event of tissue damage. Damage-associated molecular patterns (DAMPs), which are released from damaged or dying cells in noninfectious inflammations such as burn or trauma, directly or indirectly promote inflammation. (Tanaka T, Narazaki M, Kishimoto T. IL-6 in inflammation, immunity, and disease. Cold Spring Harb Perspect Biol. 2014 Sep. 4; 6 (10): a016295. doi: 10.1101/cshperspect.a016295. PMID: 25190079; PMCID: PMC4176007.)

In some embodiments, the immunomodulating agent is an IL-6 inhibitor. There are 2 classes of Food and Drug Administration (FDA)-approved IL-6 inhibitors: anti-IL-6 receptor monoclonal antibodies (mAbs) (e.g., tocilizumab, sarilumab) and anti-IL-6 mAbs (e.g., siltuximab). In some embodiments, the immunomodulating agent is an anti-IL-6 receptor antibody. In some embodiments, the immunomodulating agent is tocilizumab (Actemra®, see U.S. Pat. No. 5,888,510, US Application 20220306755A1). Tocilizumab is a recombinant humanized anti-IL-6 receptor mAb that is approved by the FDA for use in patients with rheumatologic disorders and cytokine release syndrome induced by chimeric antigen receptor T cell therapy. Tocilizumab can be administered as an intravenous (IV) infusion or an SQ injection. The IV formulation should be used to treat cytokine release syndrome. In some embodiments, the immunomodulating agent is sarilumab. Sarilumab is a recombinant humanized anti-IL-6 receptor mAb that is approved by the FDA for use in patients with rheumatoid arthritis. It is available as an SQ formulation and is not approved for the treatment of cytokine release syndrome. In some embodiments, the immunomodulating agent is an anti-IL-6 antibody. In some embodiments, the immunomodulating agent is siltuximab. Siltuximab is a recombinant human-mouse chimeric mAb that binds IL-6 and is approved by the FDA for use in patients with multicentric Castleman disease. Siltuximab prevents the binding of IL-6 to both soluble and membrane-bound IL-6 receptors, inhibiting IL-6 signaling. Siltuximab is administered as an IV infusion. Further information about tocilizumab, sarilumab, and siltuximab can be found in their respective Prescribing Information or drug labels.

In some embodiments, the immunomodulating agent reduces the expression interleukin-6 in a cell by interfering IL-6 gene transcription or translation. In some embodiments, the immunomodulating agent knocks out or knocks down the IL-6 gene in a cell. In some embodiments, the immunomodulating agent is an RNA interference (RNAi), an antisense oligonucleotide, or a small interfering RNA (siRNA), which specifically target the mRNA transcribed from the IL-6 gene.

In some embodiments of the composition disclosed herein, the therapeutic agent is an antibody. In some embodiments, the therapeutic agent is an antibody or an antigen-binding fragment thereof. As used herein, “antibody” refers to any immunoglobulin (Ig) molecules, including but not limited to, monoclonal antibody, human antibody, non-human antibody, llama antibody, humanized antibody, chimeric antibody, single domain antibody, antibody fragments, antigen binding fragment, bispecific antibody, multispecific antibody, multimeric antibody, single chain antibody, single chain variable fragment (scFv), or any functional fragment, mutant, variant, or derivation thereof, that specifically binds to or interacts with at least one particular antigen. In humans and most mammals, an antibody unit typically consists of four polypeptide chains: two identical heavy chains and two identical light chains connected by disulfide bonds. Light chains consist of one variable domain VL and one constant domain CL, while heavy chains contain one variable domain VH and three to four constant domains, e.g., CH1, CH2, CH3. Immunoglobulins can be assigned to five major classes, namely IgA, IgD, IgE, IgG, and IgM, depending on the heavy chain constant domain amino acid sequence. IgA and IgG are typically further sub-classified to IgA1, IgA2, IgG1, IgG2, IgG3, and IgG4. Antibody light chains of vertebrate species can be assigned to one of two distinct types, namely kappa (κ) and lambda (λ), based on the amino acid sequences of their constant domains.

As used herein, “antigen binding fragment” or “antibody fragment” refers to a portion of an immunoglobulin molecule that retains the heavy chain and/or the light chain antigen binding site, such as a heavy chain complementarity determining region, a light chain complementarity determining region, a heavy chain variable region (VH), or a light chain variable region (VL). Antibody fragment includes, but not limited to, a Fab fragment (a monovalent fragment consisting of the VL or the VH), a F(ab)2 fragment (a bivalent fragment comprising two Fab fragments linked by a disulfide bridge at the hinge region), a Fd fragment consisting of the VH and CH1 domains, a Fv fragment consisting of the VL and VH domains of a single arm of an antibody, a dAb fragment, which consists of a VH domain, or a variable domain (VHH) from, e.g., human or camelid origin. These antibody fragments can be obtained using well known techniques. VHH refers to an antigen binding fragment of heavy chain only antibodies.

In some embodiments, the antibody comprises a constant region which is engineered to improve the half-life of the antibody, e.g., the in vivo half-life of the antibody. In some embodiments, the constant region is engineered with the M252Y/S254T/T256E (YTE) mutation combination, as described in U.S. Pat. No. 7,083,784, and Dall'Acqua, William F., Peter A. Kiener, and Herren Wu. “Properties of human IgG1s engineered for enhanced binding to the neonatal Fc receptor (FcRn).” Journal of Biological Chemistry 281.33 (2006): 23514-23524. See also U.S. Pat. No. 7,658,921. The numbering is according to EU numbering as reported in Kabat, E. A., Wu, T. T., Perry, H. M., Gottesman, K. S., and Foeller. (1991) Sequences of Proteins of Immunological Interest, U.S. Public Health Service, National Institutes of Health, Washington, D.C. For example, Synagis is an approved antibody with the YTE mutations. (Domachowske, Joseph B., et al. “Safety, tolerability and pharmacokinetics of MEDI8897, an extended half-life single-dose respiratory syncytial virus prefusion F-targeting monoclonal antibody administered as a single dose to healthy preterm infants.” The Pediatric infectious disease journal 37.9 (2018): 886.)

In some embodiments, the antibody comprises a constant region which is engineered as described in U.S. Pat. No. 8,088,376, Ko et al., or Lee et al. (Ko, Sanghwan, Migyeong Jo, and Sang Taek Jung. “Recent achievements and challenges in prolonging the serum half-lives of therapeutic IgG antibodies through Fc engineering.” BioDrugs 35.2 (2021): 147-157; Lee, Chang-Han, et al. “An engineered human Fc domain that behaves like a pH-toggle switch for ultra-long circulation persistence.” Nature communications 10.1 (2019): 1-11.)

### Methods

In an aspect, the present disclosure provides a method comprising administering to a subject any one of the compositions or components of the kits described herein.

In an aspect, the present disclosure provides a method comprising administering to a subject a product comprising an mRNA construct encoding a therapeutic agent, e.g., a lipid nanoparticle encapsulating the mRNA construct, and administering to the subject an immunomodulating agent.

In some embodiments of the method disclosed herein, the immunomodulating agent reduces neutrophil expansion.

In some embodiments of the method disclosed herein, the immunomodulating agent reduces lymphocyte trafficking.

In some embodiments of the method disclosed herein, the immunomodulating agent reduces monocyte trafficking.

In some embodiments of the method disclosed herein, the immunomodulating agent blocks the function of interleukin-6 (IL-6) function and/or reduces the expression interleukin-6 (IL-6) in a cell. In some embodiments, the immunomodulating agent is an IL-6 inhibitor. In some embodiments, the immunomodulating agent is an anti-IL-6 receptor antibody. In some embodiments, the immunomodulating agent is tocilizumab (Actemra®, see U.S. Pat. No. 5,888,510, US Application No. 20220306755A1). In some embodiments, the immunomodulating agent is sarilumab. In some embodiments, the immunomodulating agent is an anti-IL-6 antibody. In some embodiments, the immunomodulating agent is siltuximab.

In some embodiments, the immunomodulating agent reduces the expression interleukin-6 in a cell by interfering IL-6 gene transcription or translation. In some embodiments, the immunomodulating agent knocks out or knocks down the IL-6 gene in a cell. In some embodiments, the immunomodulating agent is an RNA interference (RNAi), an antisense oligonucleotide, or a small interfering RNA (siRNA), which specifically target the mRNA transcribed from the IL-6 gene.

In some embodiments of the method disclosed herein, the therapeutic agent is an antibody. In some embodiments, the antibody comprises a constant region which is engineered to improve the in vivo half-life of the antibody. In some embodiments, the constant region is engineered with the M252Y/S254T/T256E (YTE) mutation combination, as described in U.S. Pat. No. 7,083,784, and Dall'Acqua, William F., Peter A. Kiener, and Herren Wu. “Properties of human IgG1s engineered for enhanced binding to the neonatal Fc receptor (FcRn).” Journal of Biological Chemistry 281.33 (2006): 23514-23524. See also U.S. Pat. No. 7,658,921. The numbering is according to EU numbering as reported in Kabat, E. A., Wu, T. T., Perry, H. M., Gottesman, K. S., and Foeller. (1991) Sequences of Proteins of Immunological Interest, U.S. Public Health Service, National Institutes of Health, Washington, D.C. For example, Synagis is an approved antibody with the YTE mutations. (Domachowske, Joseph B., et al. “Safety, tolerability and pharmacokinetics of MEDI8897, an extended half-life single-dose respiratory syncytial virus prefusion F-targeting monoclonal antibody administered as a single dose to healthy preterm infants.” The Pediatric infectious disease journal 37.9 (2018): 886.)

In some embodiments, the antibody comprises a constant region which is engineered as described in U.S. Pat. No. 8,088,376, Ko et al., or Lee et al. (Ko, Sanghwan, Migyeong Jo, and Sang Taek Jung. “Recent achievements and challenges in prolonging the serum half-lives of therapeutic IgG antibodies through Fc engineering.” BioDrugs 35.2 (2021): 147-157; Lee, Chang-Han, et al. “An engineered human Fc domain that behaves like a pH-toggle switch for ultra-long circulation persistence.” Nature communications 10.1 (2019): 1-11.)

In some embodiments of the method disclosed herein, the mRNA product, e.g., the lipid nanoparticle encapsulating the mRNA construct, and the immunomodulating agent are administered in the same composition, or in separate compositions simultaneously or sequentially.

In some embodiments of the method disclosed herein, the mRNA product, e.g., the lipid nanoparticle encapsulating the mRNA construct, and the immunomodulating agent are administered sequentially (e.g., within an interval of several hours or several minutes). For example, the administration of the mRNA product (e.g., the lipid nanoparticle encapsulating the mRNA construct) can be before, at the same time, or after the administration of the immunomodulating agent. In some embodiments, multiple doses of the immunomodulating agent can be administered with one dose of the mRNA product. For example, one dose of the immunomodulating agent can be administered before one dose of the mRNA product, and another dose of the immunomodulating agent can be administered after the one dose of the mRNA product.

In some embodiments of the method disclosed herein, the method further comprises measuring blood cell count, neutrophil count, circulating lymphocytes count, circulating monocytes count, and/or IL-6 level in the subject. In some embodiments, the measuring is conducted before and after administration of the mRNA product (e.g., the lipid nanoparticle encapsulating the mRNA construct) and/or the immunomodulating agent.

The compositions of the present invention may be administered and dosed in accordance with current medical practice, taking into account the clinical condition of the subject, the site and method of administration, the scheduling of administration, the subjects age, sex, body weight and other factors relevant to clinicians of ordinary skill in the art. In some embodiments, the composition described herein is administered to the subject in need thereof at an effective amount. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein production.

Suitable routes of administration include, for example, oral, rectal, vaginal, transmucosal, pulmonary including intratracheal or inhaled, or intestinal administration, parenteral delivery, including intramuscular, subcutaneous, intramedullary injections, as well as intrathecal, direct intraventricular, intravenous, intraperitoneal, intranasal, or intraocular injections. In some embodiments of the methods described herein, the mRNA product, e.g., the LNP-mRNA, and the immunomodulating agents are administered via intravenous injection.

Alternately, the compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example, compositions can be provided ill lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present invention complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

mRNA

The mRNA recited in the present disclosure may encode any protein of interest. In some embodiments, the mRNA encodes a pathogenic antigen, a tumor antigen, an antibody, or some other therapeutic protein.

In some embodiments, the mRNA encodes a protein or a peptide, which comprises a pathogenic antigen or a fragment, variant or derivative thereof. Such pathogenic antigens are derived from pathogenic organisms, in particular bacterial, viral or protozoo logical (multicellular) pathogenic organisms, which evoke an immunological reaction in a subject, in particular a mammalian subject, more particularly a human. More specifically, pathogenic antigens are preferably surface antigens, e.g., proteins (or fragments of proteins, e.g., the exterior portion of a surface antigen) located at the surface of the virus or the bacterial or protozoological organism.

Pathogenic antigens are peptide or protein antigens preferably derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57: H7, 01 1 1 and O1 04: H4, Fasciola hepatica and Fasciola gigantica, FFI prion, Fi larioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papil lomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Pol iovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis. In this context particularly preferred are antigens from the pathogens selected from Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In some embodiments, the mRNA encodes a protein or a peptide, which comprises a peptide or protein comprising a tumour antigen, a fragment, variant or derivative of said tumour antigen, preferably, wherein the tumour antigen is a melanocyte-specific antigen, a cancer-testis antigen or a tumour-specific antigen, preferably a CT-X antigen, a non-X CT-antigen, a binding partner for a CT-X antigen or a binding partner for a non-X CT-antigen or a tumour-specific antigen, more preferably a CT-X antigen, a binding partner for a non-X CT-antigen or a tumour-specific antigen or a fragment, variant or derivative of said tumour antigen; and wherein each of the nucleic acid sequences encodes a different peptide or protein; and wherein at least one of the nucleic acid sequences encodes for 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 1 9-9, CA72-4, CA125, calreticulin, CAMEL, CASP-8/m, cathepsin B, cathepsin L, CD1 9, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R1 71, HLA-A1 1/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1 R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B1 7, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1 R, M-CSF, ME1/m, mesothelin, MG50/PXDN, MMP1 1, MN/CA IX-antigen, MRP-3, MUC-1, MUC-2, MUM-1/m, MUM-2/m, MUM-3/m, myosin class 1/m, NA88-A, N-acetylglucosaminyltransferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, pi 5, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and a immunoglobulin idiotype of a lymphoid blood cell or a T cell receptor idiotype of a lymphoid blood cell, or a fragment, variant or derivative of said tumour antigen; preferably survivin or a homologue thereof, an antigen from the MAGE-family or a binding partner thereof or a fragment, variant or derivative of said tumour antigen. Particularly preferred in this context are the tumour antigens NY-ESO-1, 5T4, MAGE-C1, MAGE-C2, Survivin, Muc-1, PSA, PSMA, PSCA, STEAP and PAP.

In some embodiments, the mRNA encodes a protein or a peptide, which comprises a therapeutic protein or a fragment, variant or derivative thereof. Therapeutic proteins as defined herein are peptides or proteins, which are beneficial for the treatment of any inherited or acquired disease or which improves the condition of an individual. Particularly, therapeutic proteins play an important role in the creation of therapeutic agents that could modify and repair genetic errors, destroy cancer cells or pathogen infected cells, treat immune system disorders, treat metabolic or endocrine disorders, among other functions. For instance, Erythropoietin (EPO), a protein hormone can be utilized in treating patients with erythrocyte deficiency, which is a common cause of kidney complications. Furthermore, adjuvant proteins, therapeutic antibodies are encompassed by therapeutic proteins and also hormone replacement therapy which is e.g., used in the therapy of women in menopause. In more recent approaches, somatic cells of a patient are used to reprogram them into pluripotent stem cells, which replace the disputed stem cell therapy. Also, these proteins used for reprogramming of somatic cells or used for differentiating of stem cells are defined herein as therapeutic proteins. Furthermore, therapeutic proteins may be used for other purposes, e.g., wound healing, tissue regeneration, angiogenesis, etc. Furthermore, antigen-specific B cell receptors and fragments and variants thereof are defined herein as therapeutic proteins.

Therefore therapeutic proteins can be used for various purposes including treatment of various diseases like e.g. infectious diseases, neoplasms (e.g. cancer or tumour diseases), diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired.

In this context, particularly preferred therapeutic proteins which can be used inter alia in the treatment of metabolic or endocrine disorders are selected from (in brackets the particular disease for which the therapeutic protein is used in the treatment): Acid sphingomyelinase (Niemann-Pick disease), Adipotide (obesity), Agalsidase-beta (human galactosidase A) (Fabry disease; prevents accumulation of lipids that could lead to renal and cardiovascular complications), Alglucosidase (Pompe disease (glycogen storage disease type II)), alpha-galactosidase A (alpha-GAL A, Agalsidase alpha) (Fabry disease), alpha-glucosidase (Glycogen storage disease (GSD), Morbus Pompe), alpha-L-iduronidase (mucopolysaccharidoses (MPS), Hurler syndrome, Scheie syndrome), alpha-N-acetylglucosaminidase (Sanfil ippo syndrome), Amphiregulin (cancer, metabolic disorder), Angiopoietin ((Ang1, Ang2, Ang3, Ang4, ANGPTL2, ANGPTL3, ANGPTL4, ANGPTL5, ANGPTL6, ANGPTL7) (angiogenesis, stabilize vessels), Betacellulin (metabolic disorder), Beta-glucuronidase (Sly syndrome), Bone morphogenetic protein BMPs (BMP1, BMP2, BMP3, BMP4, BMP5, BMP6, BMP7, BMP8a, BMP8b, BMP1 0, BMP1 5) (regenerative effect, bone-related conditions, chronic kidney disease (CKD)), CLN6 protein (CLN6 disease—Atypical Late Infantile, Late Onset variant, Early Juvenile, Neuronal Ceroid Lipofuscinoses (NCL)), Epidermal growth factor (EGF) (wound healing, regulation of cell growth, proliferation, and differentiation), Epigen (metabolic disorder), Epiregulin (metabolic disorder), Fibroblast Growth Factor (FGF, FGF-1, FGF-2, FGF-3, FGF-4, FGF-5, FGF-6, FGF-7, FGF-8, FGF-9, FGF-1 0, FGF-1 1, FGF-12, FGF-1 3, FGF-1 4, FGF-1 6, FGF-1 7, FGF-1 7, FGF-1 8, FGF-19, FGF-20, FGF-21, FGF-22, FGF-23) (wound healing, angiogenesis, endocrine disorders, tissue regeneration), Galsulphase (Mucopolysaccharidosis VI), Ghrelin (irritable bowel syndrome (IBS), obesity, Prader-Willi syndrome, type II diabetes mellitus), Glucocerebrosidase (Gaucher's disease), GM-CSF (regenerative effect, production of white blood cells, cancer), Heparin-binding EGF-like growth factor (HB-EGF) (wound healing, cardiac hypertrophy and heart development and function), Hepatocyte growth factor HGF (regenerative effect, wound healing), Hepcidin (iron metabolism disorders, Beta-thalassemia), Human albumin (Decreased production of albumin (hypoproteinaemia), increased loss of albumin (nephrotic syndrome), hypovolemia, hyperbilirubinaemia), Idursulphase (Iduronate-2-sulphatase) (Mucopolysaccharidosis II (Hunter syndrome)), Integrins α.νβ3, aVp5 and α5β1 (Bind matrix macromolecules and proteinases, angiogenesis), luduronate sulfatase (Hunter syndrome), Laronidase (Hurler and Hurler-Scheie forms of mucopolysaccharidosis I), N-acetylgalactosamine-4-sulfatase (rhASB; galsulfase, Arylsulfatase A (ARSA), Arylsulfatase B (ARSB)) (arylsulfatase B deficiency, Maroteaux-Lamy syndrome, mucopolysaccharidosis VI), N-acetylglucosamine-6-sulfatase (Sanfilippo syndrome), Nerve growth factor (NGF, Brain-Derived Neurotrophic Factor (BDNF), Neurotrophin-3 (NT-3), and Neurotrophin 4/5 (NT-4/5) (regenerative effect, cardiovascular diseases, coronary atherosclerosis, obesity, type 2 diabetes, metabolic syndrome, acute coronary syndromes, dementia, depression, schizophrenia, autism, Rett syndrome, anorexia nervosa, bulimia nervosa, wound healing, skin ulcers, corneal ulcers, Alzheimer's disease), Neuregulin (NRG1, NRG2, NRG3, NRG4) (metabolic disorder, schizophrenia), Neuropilin (NRP-1, NRP-2) (angiogenesis, axon guidance, cel 1 survival, migration), Obestatin (irritable bowel syndrome (IBS), obesity, Prader-Willi syndrome, type II diabetes mellitus), Platelet Derived Growth factor (PDGF (PDFF-A, PDGF-B, PDGF-C, PDGF-D) (regenerative effect, wound healing, disorder in angiogenesis, Arteriosclerosis, Fibrosis, cancer), TGF beta receptors (endoglin, TGF-beta 1 receptor, TGF-beta 2 receptor, TGF-beta 3 receptor) (renal fibrosis, kidney disease, diabetes, ultimately end-stage renal disease (ESRD), angiogenesis), Thrombopoietin (THPO) (Megakaryocyte growth and development factor (MGDF)) (platelets disorders, platelets for donation, recovery of platelet counts after myelosuppressive chemotherapy), Transforming Growth factor (TGF (TGF-alpha, TGF-beta (TGFbeta1, TGFbeta2, and TGFbeta3))) (regenerative effect, wound healing, immunity, cancer, heart disease, diabetes, Marfan syndrome, Loeys-Dietz syndrome), VEGF (VEGF-A, VEGF-B, VEGF-C, VEGF-D, VEGF-E, VEGF-F und PIGF) (regenerative effect, angiogenesis, wound healing, cancer, permeability), Nesiritide (Acute decompensated congestive heart failure), Trypsin (Decubitus ulcer, varicose ulcer, debridement of eschar, dehiscent wound, sunburn, meconium ileus), adrenocorticotrophic hormone (ACTH) (“Addison's disease, Small cell carcinoma, Adrenoleukodystrophy, Congenital adrenal hyperplasia, Cushing's syndrome, Nelson's syndrome, Infantile spasms), Atrial-natriuretic peptide (ANP) (endocrine disorders), Cholecystokinin (diverse), Gastrin (hypogastrinemia), Leptin (Diabetes, hypertriglyceridemia, obesity), Oxytocin (stimulate breastfeeding, non-progression of parturition), Somatostatin (symptomatic treatment of carcinoid syndrome, acute variceal bleeding, and acromegaly, polycystic diseases of the liver and kidney, acromegaly and symptoms caused by neuroendocrine tumors), Vasopressin (antidiuretic hormone) (diabetes insipidus), Calcitonin (Postmenopausal osteoporosis, Hypercalcaemia, Paget's disease, Bone metastases, Phantom limb pain, Spinal Stenosis), Exenatide (Type 2 diabetes resistant to treatment with metformin and a sulphonylurea), Growth hormone (GH), somatotropin (Growth failure due to GH deficiency or chronic renal insufficiency, Prader-Willi syndrome, Turner syndrome, AIDS wasting or cachexia with antiviral therapy), Insulin (Diabetes mellitus, diabetic ketoacidosis, hyperkalemia), Insulinlike growth factor 1 IGF-1 (Growth failure in children with GH gene deletion or severe primary IGF1 deficiency, neurodegenerative disease, cardiovascular diseases, heart failure), Mecasermin rinfabate, IGF-1 analog (Growth failure in children with GH gene deletion or severe primary IGF1 deficiency, neurodegenerative disease, cardiovascular diseases, heart failure), Mecasermin, IGF-1 analog (Growth failure in children with GH gene deletion or severe primary IGF1 deficiency, neurodegenerative disease, cardiovascular diseases, heart failure), Pegvisomant (Acromegaly), Pramlintide (Diabetes mellitus, in combination with insulin), Teriparatide (human parathyroid hormone residues 1-34) (Severe osteoporosis), Becaplermin (Debridement adjunct for diabetic ulcers), Dibotermin-alpha (Bone morphogenetic protein 2) (Spinal fusion surgery, bone injury repair), Histrelin acetate (gonadotropin releasing hormone; GnRH) (Precocious puberty), Octreotide (Acromegaly, symptomatic relief of VIP-secreting adenoma and metastatic carcinoid tumours), and Palifermin (keratinocyte growth factor; KGF) (Severe oral mucositis in patients undergoing chemotherapy, wound healing). These and other proteins are understood to be therapeutic, as they are meant to treat the subject by replacing its defective endogenous production of a functional protein in sufficient amounts. Accordingly, such therapeutic proteins are typically mammalian, in particular human proteins.

For the treatment of blood disorders, diseases of the circulatory system, diseases of the respiratory system, cancer or tumour diseases, infectious diseases or immunedeficiencies following therapeutic proteins may be used: Alteplase (tissue plasminogen activator; tPA) (Pulmonary embolism, myocardial infarction, acute ischaemic stroke, occlusion of central venous access devices), Anistreplase (Thrombolysis), Antithrombin III (AT-111) (Hereditary AT-III deficiency, Thromboembolism), Bivalirudin (Reduce blood-clotting risk in coronary angioplasty and heparin-induced thrombocytopaenia), Darbepoetin-alpha (Treatment of anaemia in patients with chronic renal insufficiency and chronic renal failure (+/−dialysis)), Drotrecogin-alpha (activated protein C) (Severe sepsis with a high risk of death), Erythropoietin, Epoetin-alpha, erythropoetin, erthropoyetin (Anaemia of chronic disease, myleodysplasia, anaemia due to renal failure or chemotherapy, preoperative preparation), Factor IX (Haemophilia B), Factor Vila (Haemorrhage in patients with haemophilia A or B and inhibitors to factor VIII or factor IX), Factor VIII (Haemophilia A), Lepirudin (Heparin-induced thrombocytopaenia), Protein C concentrate (Venous thrombosis, Purpura fulminans), Reteplase (deletion mutein of tPA) (Management of acute myocardial infarction, improvement of ventricular function), Streptokinase (Acute evolving transmural myocardial infarction, pulmonary embolism, deep vein thrombosis, arterial thrombosis or embolism, occlusion of arteriovenous cannula), Tenecteplase (Acute myocardial infarction), Urokinase (Pulmonary embolism), Angiostatin (Cancer), Anti-CD22 immunotoxin (Relapsed CD33+acute myeloid leukaemia), Denileukin diftitox (Cutaneous T-cell lymphoma (CTCL)), Immunocyanin (bladder and prostate cancer), MPS (Metallopanstimulin) (Cancer), Aflibercept (Non-small cell lung cancer (NSCLC), metastatic colorectal cancer (mCRC), hormone-refractory metastatic prostate cancer, wet macular degeneration), Endostatin (Cancer, inflammatory diseases like rheumatoid arthritis as well as Crohn's disease, diabetic retinopathy, psoriasis, and endometriosis), Collagenase (Debridement of chronic dermal ulcers and severely burned areas, Dupuytren's contracture, Peyronie's disease), Human deoxy-ribonuclease I, dornase (Cystic fibrosis; decreases respiratory tract infections in selected patients with FVC greater than 40% of predicted), Hyaluronidase (Used as an adjuvant to increase the absorption and dispersion of injected drugs, particularly anaesthetics in ophthalmic surgery and certain imaging agents), Papain (Debridement of necrotic tissue or liquefication of slough in acute and chronic lesions, such as pressure ulcers, varicose and diabetic ulcers, burns, postoperative wounds, pi lonidal cyst wounds, carbuncles, and other wounds), L-Asparaginase (Acute lymphocytic leukaemia, which requires exogenous asparagine for proliferation), Peg-asparaginase (Acute lymphocytic leukaemia, which requires exogenous asparagine for proliferation), Rasburicase (Paediatric patients with leukaemia, lymphoma, and solid tumours who are undergoing anticancer therapy that may cause tumour lysis syndrome), Human chorionic gonadotropin (HCG) (Assisted reproduction), Human follicle-stimulating hormone (FSH) (Assisted reproduction), Lutropin-alpha (Infertility with luteinizing hormone deficiency), Prolactin (Hypoprolactinemia, serum prolactin deficiency, ovarian dysfunction in women, anxiety, arteriogenic erectile dysfunction, premature ejaculation, oligozoospermia, asthenospermia, hypofunction of seminal vesicles, hypoandrogenism in men), alpha-1-Proteinase inhibitor (Congenital antitrypsin deficiency), Lactase (Gas, bloating, cramps and diarrhoea due to inabi lity to digest lactose), Pancreatic enzymes (lipase, amylase, protease) (Cystic fibrosis, chronic pancreatitis, pancreatic insufficiency, post-Billroth II gastric bypass surgery, pancreatic duct obstruction, steatorrhoea, poor digestion, gas, bloating), Adenosine deaminase (pegademase bovine, PEG-ADA) (Severe combined immunodeficiency disease due to adenosine deaminase deficiency), Abatacept (Rheumatoid arthritis (especially when refractory to TNFalpha inhibition)), Alefacept (Plaque Psoriasis), Anakinra (Rheumatoid arthritis), Etanercept (Rheumatoid arthritis, polyarticular-course juvenile rheumatoid arthritis, psoriatic arthritis, ankylosing spondylitis, plaque psoriasis, ankylosing spondylitis), Interleukin-1 (IL-1) receptor antagonist, Anakinra (inflammation and cartilage degradation associated with rheumatoid arthritis), Thymulin (neurodegenerative diseases, rheumatism, anorexia nervosa), TNF-alpha antagonist (autoimmune disorders such as rheumatoid arthritis, ankylosing spondylitis, Crohn's disease, psoriasis, hidradenitis suppurativa, refractory asthma), Enfuvirtide (HIV-1 infection), and Thymosin <x1 (Hepatitis B and C). (In brackets is the particular disease for which the therapeutic protein is used in the treatment). See WO2016107877.

In some embodiments, the LNP comprises mRNA molecules which encode more than one protein. In some embodiments, the LNP comprises a first mRNA, and a second nucleic acid. In some embodiments, co-delivery and/or co-administration of the second nucleic acid facilitates and/or enhances the function or delivery of the first mRNA.

In some embodiments, the mRNA comprises at least one modified nucleotide. In some embodiments, the modified nucleotide is selected from the group consisting of pseudouridine (Ψ), 1-methylpseudouridine (m1Ψ), 5-methyluridine (m5U), 2-thiouridine (s2U), 5-methylcytidine (m5C), and N6-methyladenosine (m6A). As used herein, the term “modified nucleotide” includes any synthetic nucleotide and nucleotide analogue, and any naturally existing nucleotide other than adenine, adenine, guanine, thymine, or uracil. Exemplary nucleotide modification includes 2′-O-methylation (Nm) and conversion of uridine to pseudouridine (Ψ). Both Nm and Ψ modifications have the potential to stabilize RNA folding domains. Methylation of 2′-OH sites endows a nucleotide with greater hydrophobicity, protects against nucleolytic attack and stabilizes helices, and thus can benefit inter- or intra-molecular interactions. Pseudouridine exerts a significant rigidifying influence on the sugar-phosphate backbone and enhances base stacking. In addition, Ψ provides an additional donor site for hydrogen-bond formation, which can stabilize RNA-RNA or RNA-protein interactions (Baillieu et al., Nucleic Acids Res. 2009).

In some embodiments, the mRNA is codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA. Codon optimization tools, algorithms and services are known in the art, non-limiting examples include services from GeneArt (Life Technologies) and/or DNA2.0 (Menlo Park Calif.).

In some embodiments, the mRNA comprises 5′-untranslated region (UTR) and 3′-UTR. In some embodiments, the mRNA further comprises polyA sequence at 3′ end and/or 5′-cap. Untranslated region (UTR) refers to the region flanking the protein coding sequence on either the 3′ side or the 5′ side on an RNA. It is called 5′-UTR if it is upstream of the protein coding sequence (i.e., on the 5′ side). It is called 3′-UTR if it is downstream of the protein coding sequence (i.e., on the 3′ side). UTRs are important regulatory elements with a strong impact on the post-transcriptional regulation of gene expression. The numerous roles that UTRs play include (i) regulation of mRNA export from the nucleus, (ii) regulation of translation efficiency, (iii) orchestration of subcellular localization, and (iv) mRNA stability. Together with the complex of different RNA-interacting factors, UTRs regulate mRNA stability, export to the cytoplasm, sub-cellular localization, and translation efficiency to influence the total amount of synthesized protein. The 5′-UTR is mainly involved in translation of its downstream open reading frame. While the function of the 3′-UTR is to maintain mRNA stability.

The poly(A) tail plays an important role in maintaining mRNA stability and translation efficiency. mRNA stability can be improved by inhibiting exonuclease-mediated mRNA degradation. The poly(A) tail can also bind to multiple poly(A)-binding proteins (PABPs) while working synergistically with 5′ m7G cap sequences to regulate translational efficiency. Polyadenylation can be done by traditional enzymatic polyadenylation, adding the poly(A) tail to the 3′ end of mRNA, or by designing a fixed-length poly(A) sequence on a DNA template and transcribing the resulting length-controllable poly(A) tail. The poly(A) tail is a long chain of adenine nucleotides that is added to the 3′ end of a mRNA molecule. In some embodiments, the length of the poly(A) tail is at least 80, 90, 100, 150, 200, 250, 300, 350, 400, 450 or 500 nucleotides. In some embodiments, the length of the poly(A) tail is adjusted to control the stability of the mRNA molecule disclosed herein. For example, since the length of the poly(A) can influence the half-life of the mRNA molecule, the length of the poly(A) tail can be adjusted to modify the level of resistance of the mRNA to nucleases and thereby control the time course of protein expression. 5′ cap refers to a specially altered nucleotide on the 5′ end of the RNA sequence. According to the degree of methylation, three main cap structures are possible: cap 0, cap 1, and cap 2. A cap 0 structure is the most elementary, namely m7GpppNp; however, an mRNA of cap 0 is likely to be recognized as exogenous RNA by the host, which could stimulate the innate immune response of the host and ultimately trigger inflammatory responses. A cap1 structure (m7GpppN1mp) has a methylated 2′-OH on the first nucleotide connecting the 5′ end of the mRNA to the cap. Since the cap1 structure has only been described to date in eukaryotic mRNAs, it can be used as a signature of self-RNA, thus reducing the activation of pattern recognition receptor (PRR) and consequently improving translation efficiency of mRNA in vivo. Lastly, cap2 (m7GpppN1mpN2mp) has a methylated 2′-OH on both the first and second nucleotides that connect the 5′ end of the mRNA to the cap, and methylation improves mRNA translation efficiency. In some embodiments, the 5′ cap has a cap1 structure. In some embodiments, the 5′ cap is methylated, e.g., m7GpppN, wherein N is the 5′ terminal nucleotide of the nucleic acid carrying the 5′ cap. In some embodiments, the 5′ cap structure is selected from glyceryl, inverted deoxy abasic residue, 4′,5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′-phosphorothioate, phosphorodithioate, bridging methylphosphonate moiety, and non-bridging methylphosphonate moiety.

In some embodiments, the mRNA is a non-replicating mRNA. In some embodiments, the mRNA is a self-amplifying mRNA (saRNA). saRNA contains a long ORF after 5′ UTR encoding the four NSPs (nsP1, nsP2, nsP3, and nsP4) of alphavirus that functions as an mRNA capping enzyme, an NTPase/helicase/protease, a macrodomain, or an RNA-dependent RNA polymerase (RDRP), respectively. A subgenomic promoter can then be used to initiate transcription of the gene coding for the target antigen. Once in the cytoplasm of a host cell, saRNA undergoes translation by the endogenous ribosomal machinery, thereby enabling translation of nsP precursors to form an early replication complex. The positive-strand RNA is then used as a template to synthesize negative-strand RNA, which is the replication intermediate. With the cleavage of nsP precursors, a late replication complex is produced. Then, the negative-strand RNA of the replication intermediate is used as a template to synthesize a full-length positive-strand genomic RNA. At the same time, a subgenomic positive-strand RNA containing only information coding for the antigen is also synthesized. As a result, one copy of saRNA produces multiple copies of RNA transcripts by the above-described mechanism to initiate self-amplification of antigen genes in the cell.

In some embodiments, the mRNA is a circular RNA (circRNA). CircRNA is a highly stable single-stranded RNA with a covalently closed loop structure. Despite the lack of essential elements for cap-dependent translation, circRNA can be translated by adding the IRES element or m6A modification incorporated to its 5′ UTR region. (Wesselhoeft, R. Alexander, Piotr S. Kowalski, and Daniel G. Anderson. “Engineering circular RNA for potent and stable translation in eukaryotic cells.” Nature communications 9.1 (2018): 1-10.)

In some embodiments, the RNA is an in vitro transcribed (IVT) mRNA. In vitro transcription is a simple procedure that allows for template-directed synthesis of RNA molecules of any sequence from short oligonucleotides to those of several kilobases in μg to mg quantities. In some embodiments, it is based on the engineering of a template that includes a bacteriophage promoter sequence (e.g., from the T7 coliphage) upstream of the sequence of interest followed by transcription using the corresponding RNA polymerase. Techniques for in vitro transcription is well known in the art. (Beckert, Bertrand, and Benoît Masquida. “Synthesis of RNA by in vitro transcription.” Rna. Humana Press, 2011. 29-41.)

### LNP

These nanoparticles are typically composed of a mixture of lipids such as cationic lipids, ionizable lipids, phospholipids, cholesterol, PEG-lipids, and other lipids that can form a stable and protective structure around the drug. Lipid nanoparticles can be engineered to have specific properties such as size, surface charge, and stability, to achieve targeted and controlled drug delivery to specific cells or tissues in the body.

Some examples of lipids that can be used for LNP can be found in Hou, Xucheng, et al. “Lipid nanoparticles for mRNA delivery.” Nature Reviews Materials 6.12 (2021): 1078-1094. Exemplary formulations and production methods of LNP can be found in US20220378939A1, U.S. Pat. No. 8,058,069B2.

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1 Overall Study Design

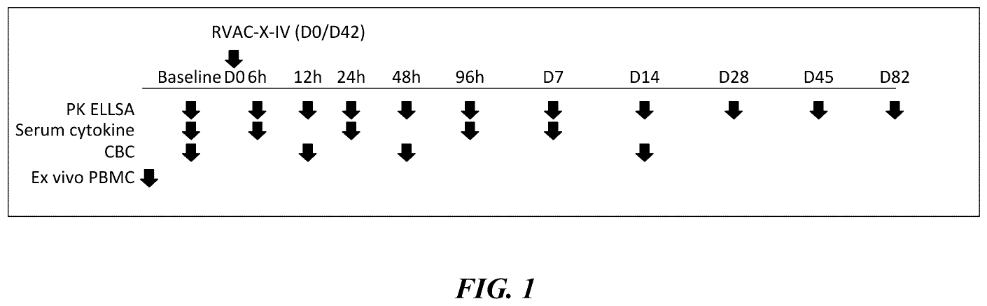

A study protocol was designed to measure differences in pharmacodynamics and pharmacokinetic response between recombinant human IgG1 and mRNA/LNP encoded human IgG1. Furthermore, the study aimed to determine the effect of half-life enhancing mutation (YTE) on the pharmacokinetic profile of the test articles. Table 1, FIG. 1.

TABLE 1

| | | | Adminis- | Number | |
|---|---|---|---|---|---|
| | | | tration | of | |
| Group | Test article | Dose | route | animals | Animal |
| 1 | RVAC-IgG-wt | 10 mg/kg | multiple | 2 | Cyno |
| | (protein) | | dose, i.v. | | |
| 2 | RVAC-IgG-YTE | 10 mg/kg | injection | 2 | Cyno |
| | (protein) | | | | |
| 3 | RVAC-IgG-wt | 2.5 mg/kg | | 2 | Cyno |
| | (mRNA-LNP) | | | | |
| 4 | RVAC-IgG-YTE | 2.5 mg/kg | | 2 | Cyno |
| | (mRNA-LNP) | | | | |

### Example 2 Screening of Cynomolgus Monkeys Using Ex Vivo Cytokine Release Assay

Blood was collected from 14 cynomolguses monkeys and PBMC isolated and cultured in 96 well plates for 24 hours prior to addition of each test article at two concentrations, 1 and 10 μg/ml. R848 (resiquimod) was used as a positive control at 1 μg/ml. PBMCs were incubated at 37° C. with the test articles for 3 days at which point the plates were spun down and sups collected for analysis.

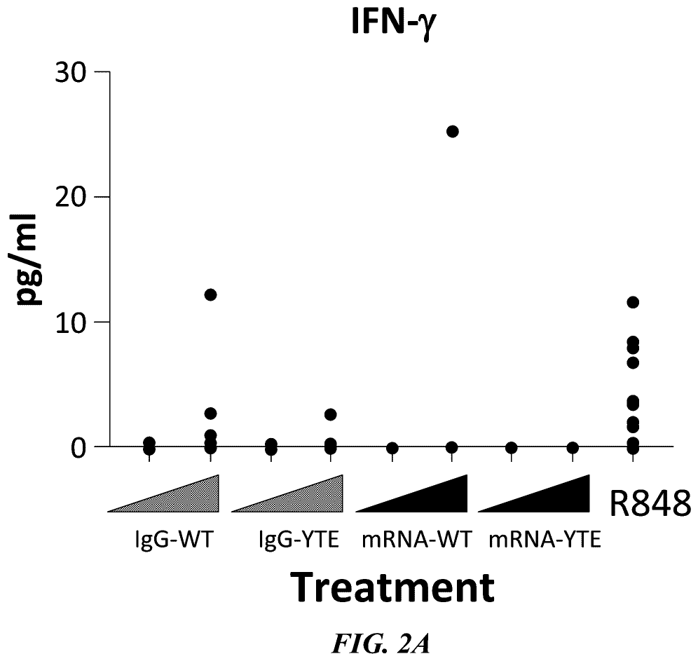

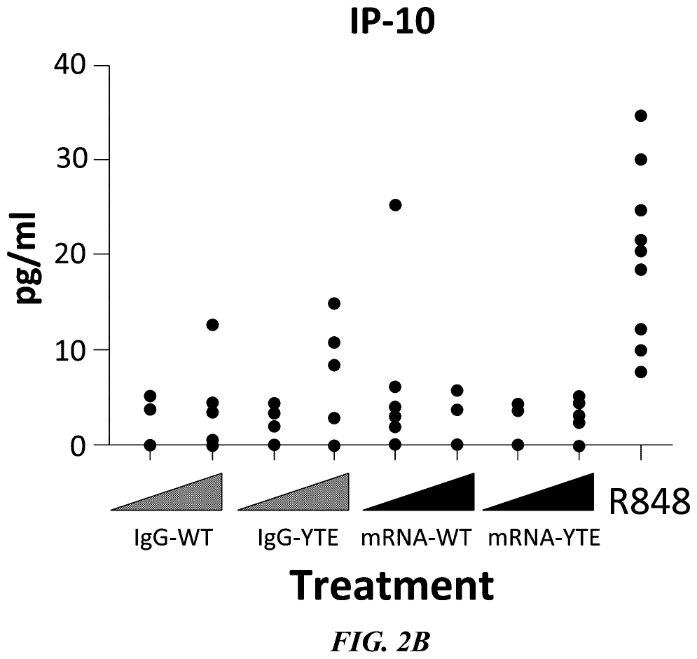

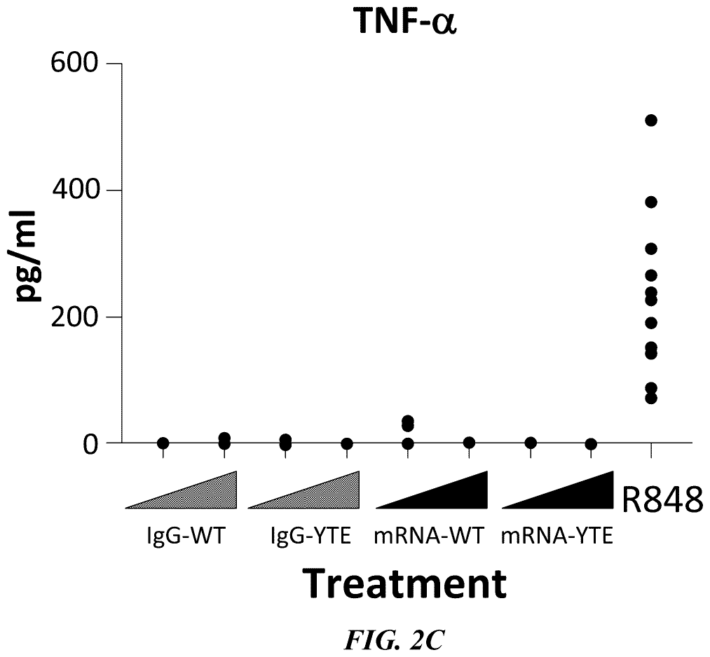

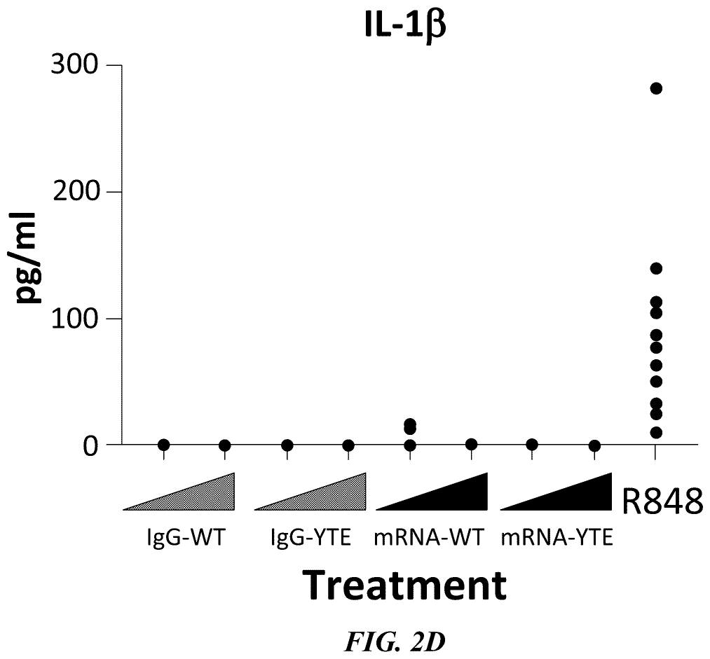

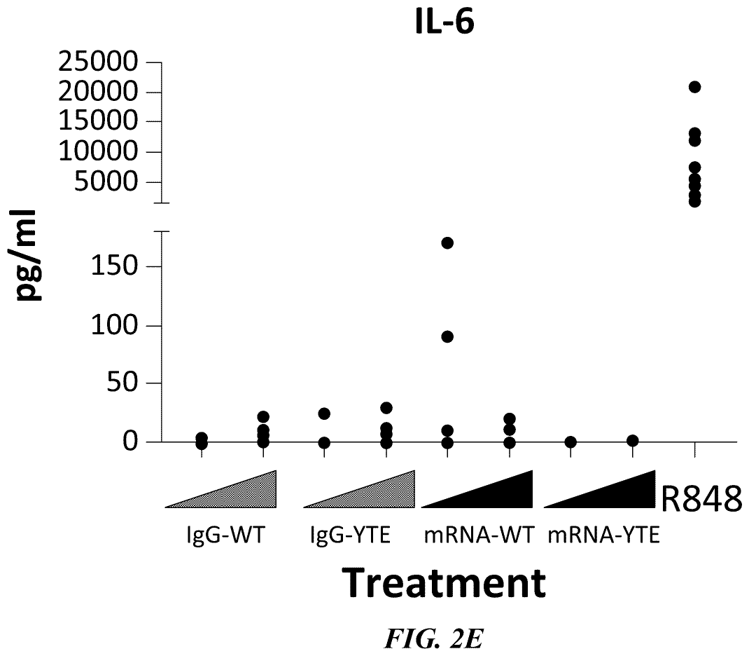

Cytokine analysis was conducted using a Luminex MagPix instrument using a multiplex kit for measurement of IFN-gamma (FIG. 2A), IP-10 (FIG. 2B), TNF-a (FIG. 2C), IL-1b (FIG. 2D), and IL-6 (FIG. 2E).

### Example 3 Measuring White Blood Cell Counts

Complete blood cell (CBC) counts were obtained for each animal pre and post dosing according to the study protocol.

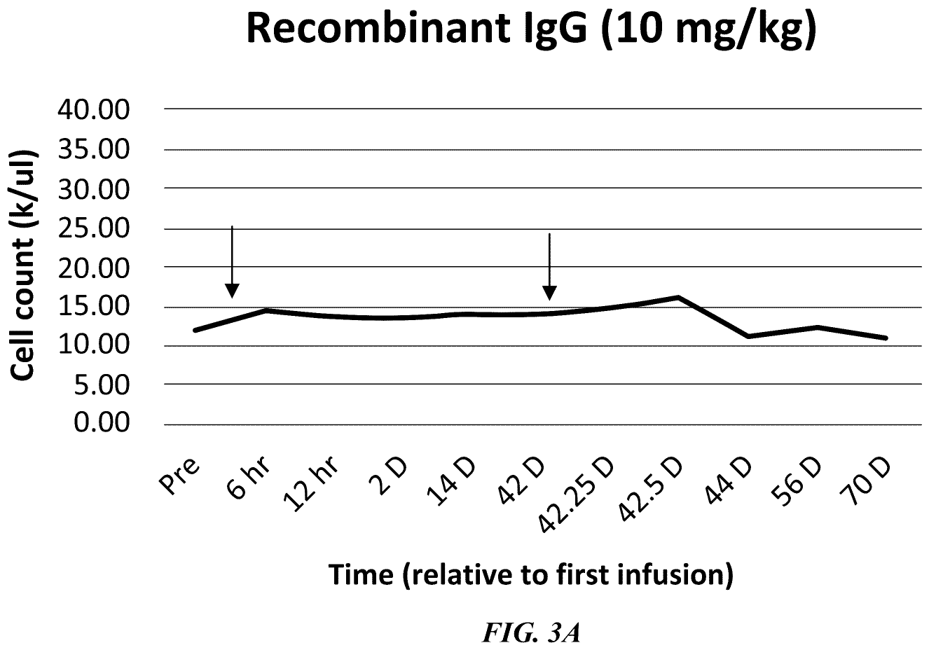

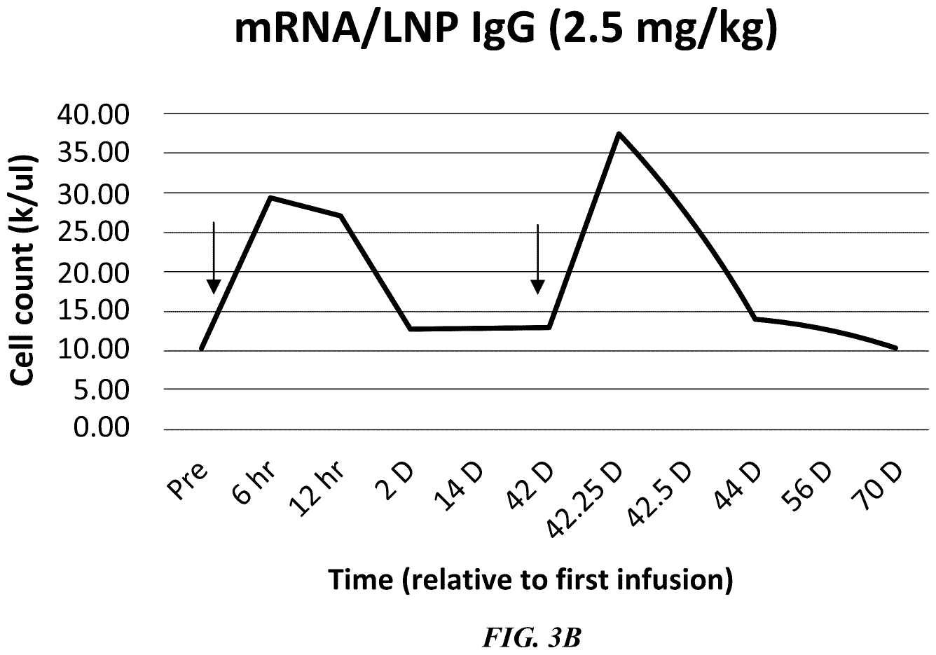

FIG. 3 shows the average counts in thousands of cells per microliter of blood (k/ul) for recombinant IgG treated animals (FIG. 3A) and mRNA-LNP IgG treated animals (FIG. 3B). Arrows indicate the relative time of dosing.

mRNA-LNP IgG treated animals show a rapid and transient increase of white blood cell counts after intravenous infusion.

### Example 4 Measuring Changes in White Blood Cells Subsets for mRNA/LNP Dosed Animals

White blood cells (WBC) counts obtained from CBC are composed primarily of three subsets of immune cells: neutrophils, lymphocytes, and monocytes. Relative measures for each immune subset were obtained from the CBC counts for each animal pre- and post-dosing according to the study protocol.

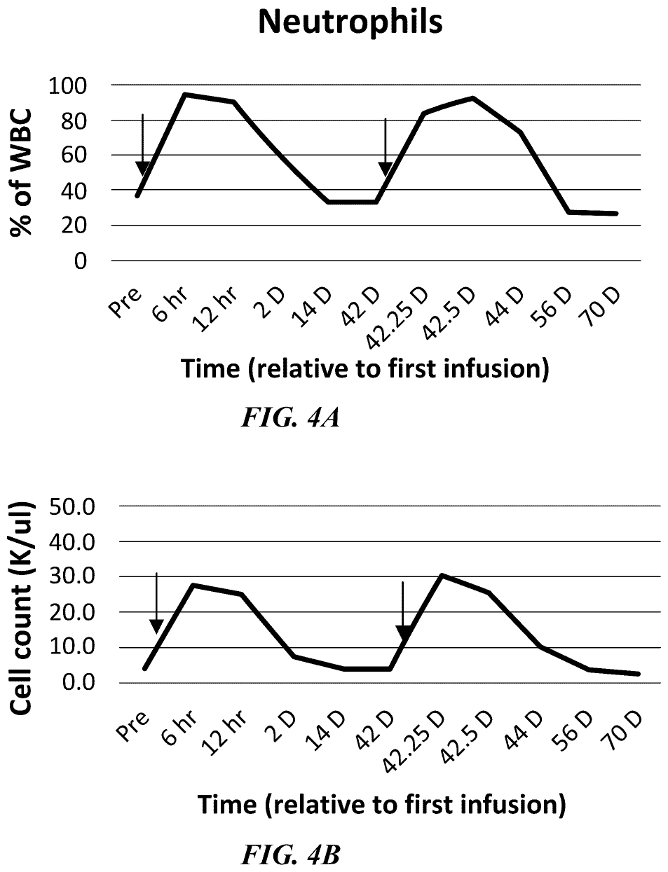

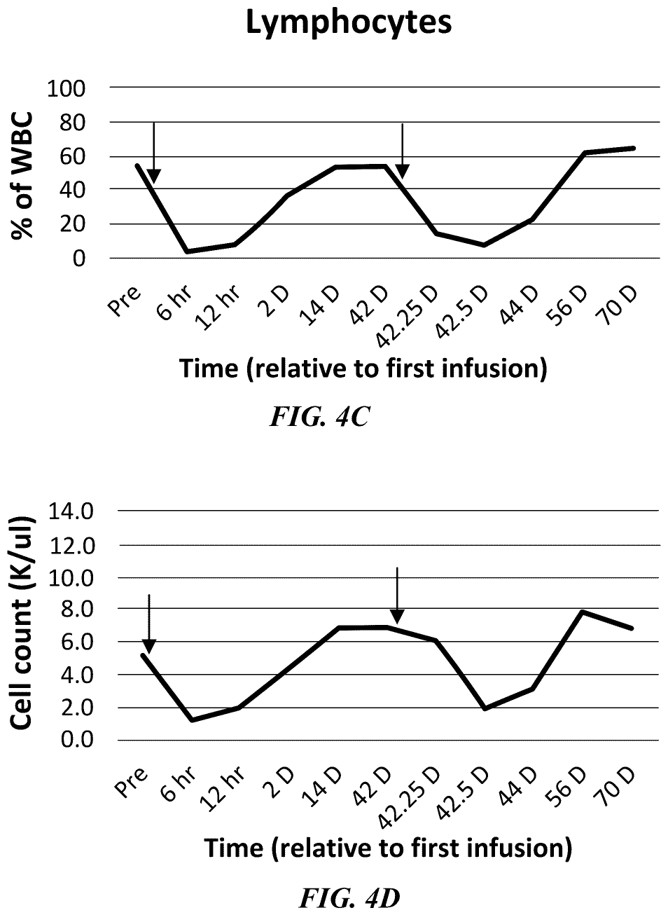

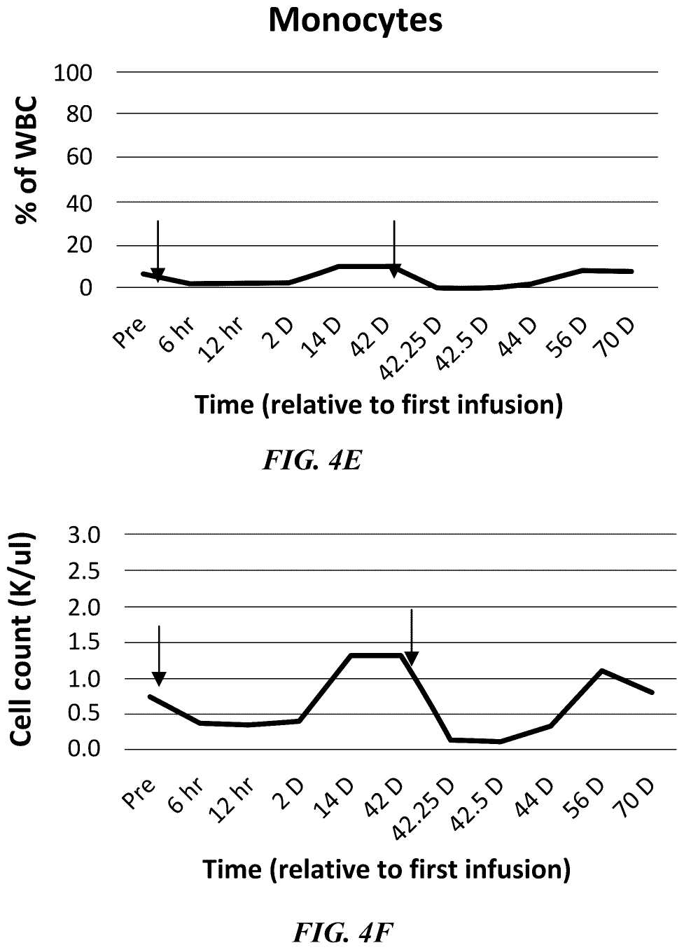

FIG. 4 shows average counts as percentage of WBC (% of WBC) and in thousands of cells per microliter of blood (K/ul) for mRNA-LNP IgG treated animals. Arrows indicate the relative time of dosing. Neutrophil as a percentage of WBC (FIG. 4A) and as total cells per microliter of blood (FIG. 4B) rise at 6 hrs. post infusion and recover to baseline by day 14 post infusion. Lymphocytes as a percentage of WBC (FIG. 4C) and as total cells per microliter of blood (FIG. 4D) drop at 6 hrs. post infusion and recover to baseline by day 14 post infusion. Monocytes as a percentage of WBC (FIG. 4E) and as total cells per microliter of blood (FIG. 4F) drop at 6 hrs. post infusion and recover to baseline by day 14 post infusion.

### Example 5 IL-6 Serum Cytokine Response

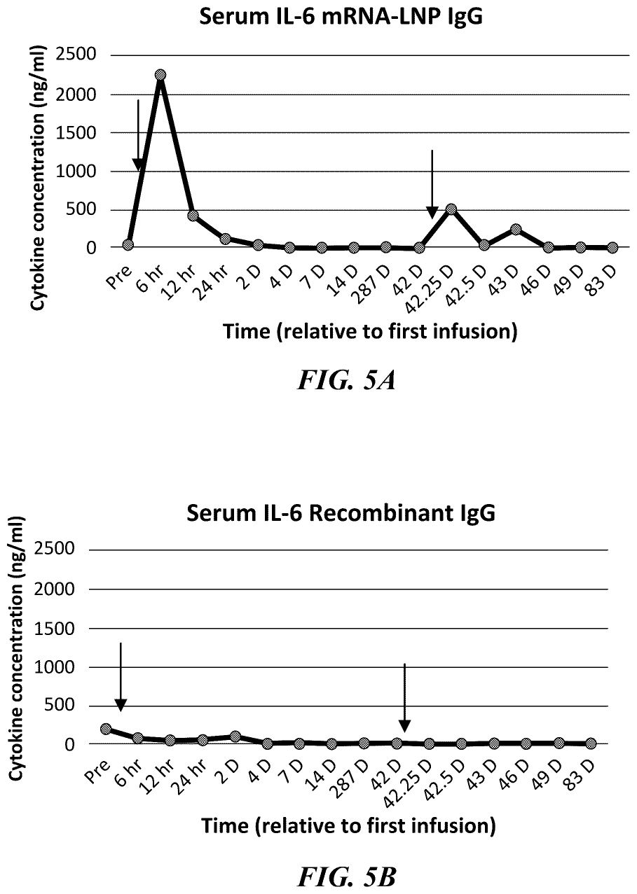

Blood was collected from cynomolgus monkeys pre- and post-dosing as indicated in the study protocol to measure serum cytokines. Cytokine analysis was conducted using Luminex MagPix instrument using multiplex kit for measurement of IFNg, IL-1b, IL-6, TNF-a, and IP-10. IL-6 serum levels showed transient increase in mRNA/LNP dosed animals (FIG. 5A) but not in recombinant IgG doses animals (FIG. 5B). IL-6 serum levels showed transient increase in mRNA/LNP dosed animals up to a peak at 6 hrs. post infusion and recovery to baseline by 48 hrs post infusion.

### Example 6 Testing the Effect of Half-Life Enhancing Mutation (YTE) on the Pharmacokinetic Profile of the Test Articles

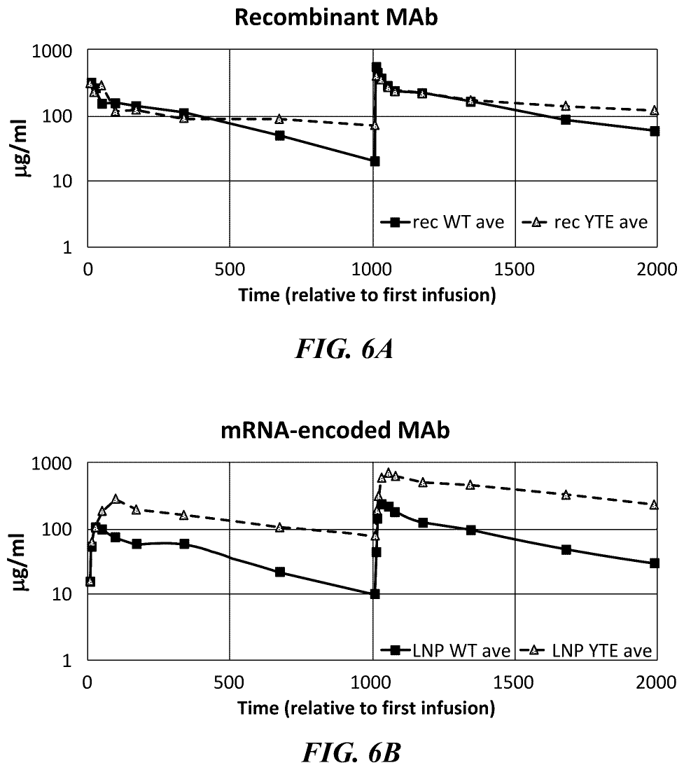

Two animals per group were dosed with 10 mg/kg WT or YTE recombinant antibody or with 2.5 mg/kg mRNA coated LNP expressing the WT or YTE antibody. mRNA was mixed in a ratio of 2:1 of Heavy chain to light chain prior to formulation. Animals were injected by IV at D=0 and D=42 and bloods collected at t=6H, 12 h, 24H, 48H, 96H, 7D, 14D, 28D. Serum hIgG titer was calculated by ELISA at the indicated times. The YTE antibody showed increased stability compared to the WT antibody when administered as a recombinant protein or mRNA-LNP. mRNA encoded antibodies showed favorable PK profile. Serum levels of antibodies are plotted against collection time for recombinant mAbs (FIG. 6A) and mRNA encoded mAbs (FIG. 6B).

The AUCₗₐₛₜ ratios:

mRNA/LNP-WT: rec-WT=0.5

mRNA/LNP-YTE: rec-YTE=1.5

Extended half-life YTE mutation conferred increased exposure.

No evidence of ADA up to day 82.

Cₘₐₓ for mRNA/LNP up to 96 hrs. post infusion.

1. A composition comprising an immunomodulating agent and a product comprising an mRNA construct encoding a therapeutic agent, wherein the mRNA construct is optionally encapsulated in a lipid nanoparticle.

2. The composition of claim 1, wherein the immunomodulating agent reduces neutrophil expansion.

3. The composition of claim 1, wherein the immunomodulating agent reduces lymphocyte trafficking.

4. The composition of claim 1, wherein the immunomodulating agent reduces monocyte trafficking.

5. The composition of claim 1, wherein the immunomodulating agent blocks the function of interleukin-6 (IL-6) function and/or reduces the expression interleukin-6 (IL-6) in a cell.

6. The composition of claim 1, wherein the therapeutic agent is an antibody.

7. The composition of claim 6, wherein the antibody comprises a constant region which is engineered to improve the half-life of the antibody.

8. A method comprising administering to a subject the composition of claim 1.

9. A method comprising administering to a subject a product comprising an mRNA construct encoding a therapeutic agent (“mRNA product”), wherein the mRNA construct is optionally encapsulated in a lipid nanoparticle, and administering to the subject an immunomodulating agent.

10. The method of claim 9, wherein the immunomodulating agent reduces neutrophil expansion.

11. The method of claim 9, wherein the immunomodulating agent reduces lymphocyte trafficking.

12. The method of claim 9, wherein the immunomodulating agent reduces monocyte trafficking.

13. The method of claim 9, wherein the immunomodulating agent blocks the function of interleukin-6 (IL-6) function and/or reduces the expression interleukin-6 (IL-6) in a cell.

14. The method of claim 9, wherein the therapeutic agent is an antibody.

15. The method of claim 14, wherein the antibody comprises a constant region which is engineered to improve the half-life of the antibody.

16. The method of claim 9, wherein the mRNA product and the immunomodulating agent are administered simultaneously or sequentially.

17. The method of claim 9, further comprising measuring blood cell count, neutrophil count, circulating lymphocytes count, circulating monocytes count, and/or IL-6 level of the subject.

18. The method of claim 17, wherein the measuring is conducted before and after administration of the mRNA product and/or the immunomodulating agent.

19. A kit comprising (a) a composition which comprises an immunomodulating agent and (b) a product comprising an mRNA construct encoding a therapeutic agent, wherein the mRNA construct is optionally encapsulated in a lipid nanoparticle.
